(19) United States
(12) Patent Application Publication
(10) Pub. No.: US 2009/0143348 A1
(43) Pub. Date: Jun. 4, 2009

(54) POLYSACCHARIDE GEL COMPOSITIONS AND METHODS FOR SUSTAINED DELIVERY OF DRUGS

(76) Inventors: Ahmet Tezel, Goleta, CA (US); Michael R. Robinson, Irvine, CA (US)

Correspondence Address: ALLERGAN, INC., 2525 DUPONT DRIVE, T2-7H, IRVINE, CA, 92612-1599, US

(21) Appl. No.: 12/323,251

(22) Filed: Nov. 25, 2008

Related U.S. Application Data
(60) Provisional application No. 60/991,524, filed on Nov. 30, 2007.

(51) Int. Cl. A61K 47/36; C08B 37/00; C08B 37/10; A61K 31/58; C08B 37/08; C08B 37/04

(52) U.S. Cl. 514/174; 536/124; 514/777

(57) ABSTRACT

Methods of producing a biocompatible polysaccharide gel composition having sustained release properties are disclosed. Also disclosed is a biocompatible polysaccharide gel composition having sustained release properties, a method of treating a disease or condition using the present biocompatible polysaccharide gel composition, and a method of controlling rate of release of at least one target solute from the biocompatible polysaccharide gel composition. Pharmaceutical compositions which include the present biocompatible polysaccharide gel composition also are disclosed.

### CROSS REFERENCE TO RELATED APPLICATIONS

This application claims the benefit of U.S. Provisional Application No. 60/991,524 filed on Nov. 30, 2007, the entirety of which is hereby incorporated by reference.

### FIELD OF THE INVENTION

Disclosed herein generally are biocompatible polysaccharide gel compositions having sustained release properties useful for cosmetic and medical applications, and products and related methods for using and making the same.

### BACKGROUND OF THE INVENTION

Polysaccharides are relatively complex carbohydrates. They are polymers made up of many monosaccharides joined together by glycosidic bonds. They are therefore large, often branched, macromolecules. Polysaccharide fillers, especially hyaluronic acid fillers have been useful in cosmetic and medical applications. These fillers have been used for example in soft tissue augmentation.

Residing in the extracellular space, hyaluronic acid functions as a space-filling, structure stabilizing, and cell protective molecule with uniquely malleable physical properties and superb biocompatibility. Hyaluronic acid matrices are extremely viscoelastic while preserving a high level of hydration. A strong correlation exists between the water content in the skin and levels of hyaluronic acid in dermal tissue. As human skin ages, there are known alterations in hyaluronic acid content and metabolism. With these changes, there is a significant deterioration in the mechanical properties of the skin. There appears to be a relationship between youthful skin and the presence of a strong hyaluronic acid network in the intercellular matrix.

Hyaluronic acid (also called hyaluronic acid or hyaluronate) is a non-sulfated glycosaminoglycan distributed widely throughout connective, epithelial, and neural tissues. It is one of the chief components of the extracellular matrix, contributes significantly to cell proliferation and migration, and may also be involved in the progression of some malignant tumors. The average 70-kg man has roughly 15 grams of hyaluronic acid in his body, one-third of which is turned over (degraded and synthesized) every day.

Hyaluronic acid is naturally found in many tissues of the body, such as skin, cartilage, and the vitreous humor. It is therefore well suited to biomedical applications targeting these tissues. The first hyaluronic acid biomedical product, Healon®, was developed in the 1970s and 1980s, and is approved for use in eye surgery (i.e., corneal transplantation, cataract surgery, glaucoma surgery and surgery to repair retinal detachment).

Hyaluronic acid is also used to treat osteoarthritis of the knee. Such treatments, called viscosupplementation, are administered as a course of injections into the knee joint and are believed to supplement the viscosity of the joint fluid, thereby lubricating the joint, cushioning the joint, and producing an analgesic effect. It has also been suggested that hyaluronic acid has positive biochemical effects on cartilage cells. However, some placebo controlled studies have cast doubt on the efficacy of hyaluronic acid injections, and hyaluronic acid is recommended primarily as a last alternative to surgery. Oral use of hyaluronic acid has been suggested. At present, there are some preliminary clinical studies that suggest that oral administration of hyaluronic acid has a positive effect on osteoarthritis.

Due to its high biocompatibility and its common presence in the extracellular matrix of tissues, hyaluronic acid also has gained popularity as a biomaterial scaffold in tissue engineering research. In some cancers, hyaluronic acid levels correlate well with malignancy and poor prognosis. Hyaluronic acid is thus often used as a tumor marker for prostate and breast cancer. It may also be used to monitor the progression of the disease. Hyaluronic acid may also be used postoperatively to induce tissue healing, notably after cataract surgery. Current models of wound healing propose that larger polymers of hyaluronic acid appear in the early stages of healing to physically make room for white blood cells, which mediate the immune response.

Therapeutic use of a hyaluronic acid or of a corticosteroid is known. Thus, hyaluronic acid (also called hyaluronan and sodium hyaluronate) formulations for both therapeutic and cosmetic use are known. Hyaluronic acid is most frequently referred to as hyluronan due to the fact that it exists in vivo as a polyanion and not in the protonated acid form. U.S. Pat. Nos. 4,636,524; 4713,448; 5,009,013, and 5,143,724 disclose particular hyaluronans or hyaluronic acids and methods for making them. Additionally, intra-articular use of a hyaluronic acid (i.e. as a viscosupplement) or of an anti-inflammatory steroid is known. See e.g. Kopp S. et al., The short-term effect of intra-articular injections of sodium hyaluronate and corticosteroid on temporomandibular joint pain and dysfunction, J Oral Maxillofac Surg June 1985; 43(6): 429-35; Grecomoro G., et al., Intra-articular treatment with sodium hyaluronate in gonarthrosis: a controlled clinical trial versus placebo, Pharmatherapeutica. 1987; 5(2):137-41; Adams M., An analysis of clinical studies of the use of crosslinked hyaluronan, hylan, in the treatment of osteoarthritis, J Rheumatol Suppl. August 1993; 39:16-8, and; Jones, A. et al., Intra-articular hyaluronic acid compared to intra-articular triamcinolone hexacetonide in inflammatory knee osteoarthritis, Osteoarthritis Cartilage. December 1995; 3(4):269-7.

Commercially available hyaluronic acid formulations include Juvederm™. (Allergan), an injectable dermal filler comprised of a cross-linked hyaluronic acid. Also known are Orthovisc®. (Anika), Durolane (Smith & Nephew), Hyalgan®. (Sanofi), Hylastan®. (Genzyme), Supartz®. (Seikagaku/Smith & Nephew)), Synvisc®. (Genzyme), Euflexxa®, (Ferring) which are used as injectable (intra-articular) hyaluronic acid viscosupplements, of various molecular weights with various degrees of cross-linking of the hyaluronic acid, for treating osteoarthritis joint pain.

Compositions for therapeutic or cosmetic use comprising a high molecular weight hyaluronic acid and one or more active agents has been disclosed. See e.g. U.S. patent application Ser. Nos. 11/039,192; 11/695,527; 11/742,350; 10/966,764; 11/354,415, and; 11/741,366.

Certain corticosteroids (such as triamcinolone) can have anti-inflammatory properties. Thus, intra-articular corticosteroids have been used to treat various joint diseases. See e.g. Zulian F., et al., Triamcinolone acetonide and hexacetonide intra-articular treatment of symmetrical joints in juvenile idiopathic arthritis: a double-blind trial, Rheum 2004; 43:1288-1291. (use of 2 mg to 80 mg of triamcinolone acetonide) and; Hertzberger-ten Cate R. et al., Intra-articular steroids in pauciarticular juvenile chronic arthritis, type I, Eur J Ped 1991; 150: 170-172 (intra-articular 20 mg triamcinolone used to treat juvenile arthritis). Triamcinolone has been used to treat joint stiffness (Clark D. et al., The influence of triamcinolone acetonide on joint stiffness in the rat, J Bone Joint Surg Am 1971; 53:1409-144).

Additionally, intramuscular steroids have been given to treat acute conditions, until the patient can be managed by use of oral steroids, such as asthma (Mancinelli L. et al., Intramuscular high-dose triamcinolone acetonide in the treatment of severe chronic asthma, West J Med November 1997:167(5); 322-329 [up to 360 mg of the triamcinolone was administered daily for three days to a patient]). Subcutaneous and intradermal administration of a steroid is not a preferred route of administration because dermal atrophy can result. When administered by intramuscular injection the risk of dermal atrophy by the steroid can be reduced by giving the injection in a deep gluteal muscle area and avoiding leakage of the steroid formulation into the dermis.

Unfortunately, there are significant drawbacks and deficiencies with known viscous formulations and with known corticosteroid formulations for peripheral use. For example, multiple (five or more) peripheral administrations of a hyaluronic acid can be required to treat a peripheral condition. Additionally, an aqueous corticosteroid formulation of triamcinolone can quickly clear (diffuse out of and/or is removed by one or more active transport mechanisms) from the site of peripheral administration. Rapid clearance can necessitate frequent re-administration (re-dosing) in order to provide an effective treatment. Additionally, therapeutic corticosteroids due to their low water solubility are typically administered as an aqueous suspension of relatively large, irregularly shaped crystals (particles). Such steroid particles can induce an inflammatory response upon administration. This may occur because macrophages present at the administration site can be unable to remove the steroid particles (by phagocytosis) which have a large morphology and irregular geometry. Indeed such particles can be toxic to macrophages and lead to cell death. The death of macrophages then leads to release of pro-inflammatory cytokines that cause both acute and chronic inflammation. Clinical examples of toxicity from particles include gouty arthritis, where urate crystals that range from 5 to 20 microns can cause arthritis. See eg. Helliwell P, Use of an objective measure of articular stiffness to record changes in finger joints after intra-articular injection of corticosteroid, Ann Rheum Dis 1997; 56: 71-73 (intra-articular corticosteroid injection can cause crystal synovitis).

Thus, it is known that macrophages are injured when phagocytosing urate crystals leading to an inflammatory response. Notably, patients treated with medication that reduces macrophage activity, such as colchicine, have a dramatic improvement in their arthritis. Another clinical example of joint deposition of large, irregularly shaped crystals that are injurious to macrophages is pseudo-gout. Here, joint inflammation is caused by deposition of calcium pyrophosphate dehydrate in patients that have hyperparathyroidism. An example of joint inflammation related to injected drug particles is crystal-induced synovitis, where 1-2% of patients that receive intra-articular injections of Lederspan, Kenalog, or other corticosteroid depot formulations, develop a post-injection exacerbation of the joint inflammation. (McCarty D., et al., Inflammatory reaction after intrasynovial injection of microcrystalline adrenocorticosteroid esters, Arthritis and Rheumatism, 7(4); 359-367 (1964) (intra-articular injection of corticosteroids crystals can cause sterile inflammation also referred to as post-injection flare). See also Selvi E. et al., Arthritis induced by corticosteroid crystals, J Rheumatology 2004; 31: 3 (osteoarthritis patient treated with intra-articular injection of 40 mg triamcinolone hexacetonide developed acute arthritis induced by the injected triamcinolone crystals). The particles in these formulations, which are on the average over 10 microns and have irregular morphology, are very similar to the urate crystals in joint of patients with gout or pseudo-gout.

A triamcinolone pharmaceutical composition available under the trade name Kenalog® (Bristol-Myers-Squibb, Princeton N.J.) has been used to treat various conditions by intramuscular or intra-articular (intrabursal use) administration. Each milliliter (ml) of Kenalog® 40 composition comprises 40 milligrams (mg) of triamcinolone acetonide, sodium chloride as a tonicity agent, 10 mg (0.99%) benzyl alcohol as a preservative, 7.5 mg (0.75%) of carboxymethylcellulose sodium and 0.4 mg (0.04%) of polysorbate 80 as resuspension aids. Benzyl alcohol preservative and/or polysorbate 80 can potentially be toxic to sensitive tissues. Thus, preservative-containing corticosteroid formulations have been linked to cases of adhesive arachnoiditis following epidural injections exacerbating a patient's back pain. See e.g. Hurst, E. W., Adhesive Arachnoiditis and Vascular Blockage caused by Detergents and Other Chemical Irritants: an Experimental Study. J. Path. Bact., 1955. 70: p. 167; DeLand, F. H., Intrathecal toxicity studies with benzyl alcohol. Toxicol Appl Pharmacol, 1973. 25(2): p. 153, and; Hetherington, N. J. and M. J. Dooley, Potential for patient harm from intrathecal administration of preserved solutions. Med J Aust, 2000. 173(3): p. 141.

Significantly, the triamcinolone acetonide in Kenalog® rapidly separates and precipitates from the remainder of the formulation. For example, if Kenalog® is left standing for as short a time as about five to ten minutes a substantial separation of a triamcinolone acetonide precipitate from the remainder of the composition occurs. Unfortunately, such rapid settling of the triamcinolone also occurs with other known saline based suspensions of triamcinolone (with or with preservatives and stabilizers). A substantially uniform suspension (which is not provided by Kenalog or other saline based suspensions of triamcinolone) would be beneficial to provide a consistent and accurate dose upon administration of the suspension. In addition, resuspension processing requires the use of the resuspension aids noted above which can affect sensitive tissues.

Additionally, administration of known formulations of a corticosteroid, such as triamcinolone can also result in an allergic or inflammatory reaction possibly due to the burst or high release rates of triamcinolone from the known formulations. As noted above such a reaction can also be due to or be exacerbated due to the large and irregular size of the insoluble corticosteroid particles administered.

Over the years, methods have been developed to achieve the delivery of a therapeutic drug to a mammal requiring pharmaceutical treatment. Biodegradable carriers are ideally biocompatible and allow desired release of target solutes or drugs. The desired release of target solutes is often sustained release. Thus, there is a need for novel biocompatible polysaccharide gel compositions which provides for sustained delivery of target solutes such as drugs and also a need for formulations for peripheral administration to treat a peripheral condition which will not have the undesirable characteristics of: presence of toxic preservatives or surfactants in the formulation; rapid release of most or all of the active agent, and that will have a longer period of residence of the active agent at the site of peripheral administration and well as comprising a non or low immunogenic formulation.

### SUMMARY OF THE INVENTION

These and other objectives are achieved by the compositions and methods of the present disclosure, which, in a broad aspect, provide novel biocompatible polysaccharide gel compositions and associated methods to achieve sustained target solute or drug delivery. In accordance with the scope and teachings of the present disclosure grafting or encapsulating target solutes or drugs into polysaccharide matrices produces biocompatible polyssacharide gel compositions which achieve controlled release. Grafting at least one target solute such as a drug onto a polysaccharide such as hyaluronic acid may be achieved by covalent linkage of the at least one target solute or drug with the polysaccharide. In a broad aspect, the covalent linkage between at least one target solute and polysaccharide may be performed by use of one or more hydroxyl and/or carboxyl groups located on a polysaccharide such as hyaluronic acid. Covalent bonds formed are stronger than non-covalent interactions which associate a drug with hyaluronic acid according to prior methods. The strong covalent bonds however may be broken, and thus release at least one target solute into the body of a patient. Bonds may be broken by reactions which sever covalent bonds an example of which is hydrolysis.

Covalent bond formation and later severing significantly improves the desired release characteristics and achieves superior sustained release. Any target solute which has the appropriate functional groups for covalent linkage may be used to bond with a polysaccharide matrix. Reactions for bond formation such as those that proceed by acid-base chemistry may be used. A skilled artisan is aware of the reactions and reaction conditions necessary to covalently link at least one target solute with a polysaccharide such as hyaluronic acid having the necessary functional groups for linkage.

A preferred hyaluronic acid (“HA”) as used in the present compositions has the following characteristics. First the HA provides an increase in viscosity but has a high shear rate, meaning that it retains syringeability through 25-30 gauge needles. Second, HA is a natural component of the extracelllular matrix of many mammalian tissues therefore providing a biocompatible viscosity inducing component. Third, the HA is a tissue adhesive so that when HA is inject into a tissue such as a muscle diffusion and migration of the HA through facial planes is minimized. See e.g. Cohen et al. Biophys J. 2003; 85: 1996-2005. A poorly adhesive polymer such as silicone can migrate through tissues. See e.g. Capozzi et al. Plast Reconstr Surg. 1978; 62:302-3. The tissue adhesion and therefore low tissue migration characteristic of a formulation which comprises HA enables the formulation to remain largely at the injection site. Thus a corticosteroid-HA formulation will have the advantageous characteristic of low diffusion out of the peripheral location, such as an intra-articular location (i.e. to treat facet joint arthritis). Additionally, a botulinum toxin-HA formulation will have the advantageous characteristic of low diffusion out of the peripheral location, such as an intramuscular location (i.e. into the small orbicularis muscle to treat hemifacial spasm). Hence, use of HA in a formulation can limit drug or biologic exposure to surrounding or adjacent non-target tissues, thereby limiting side effects (with regard to para-ocular botulinum toxin administration) such as ptosis or visual impairment.

Third, in order to have drug released from a carrier or the active agent (i.e. steroid crystals) solubilized contact with water is required. The preferred HA used provides this through an ability to become hydrated (absorb water).

Fourth, the HA used is a polymer that can be cross-linked to varying degrees, thereby permitting alteration of characteristics such as rate of HA migration for the peripheral location of administration, rate of active agent diffusion and migration out of the HA carrier.

One particular drug which may be covalently linked to polysaccharides such as hyaluronic acid and delivered to a patient as a biocompatible polysaccharide gel composition is triamcinolone acetonide. In one embodiment, the triamcinolone particles of the present gel compositions are substantially uniformly suspended with a viscosity inducing component being hyaluronic acid, or polymeric hyaluronate.

The present disclosure further generally relates to methods of producing biocompatible polysaccharide gel compositions by encapsulating at least one target solute such as a drug into porous networks of polysaccharide gels. Such encapsulation is another useful way of associating a drug to be delivered with a polysaccharide such as hyaluronic acid which may or may not be cross-linked in accordance with the scope and teachings of the present disclosure.

Yet another aspect of the present disclosure relates to methods of treating a disease or condition by administering a therapeutically effective amount of the biocompatible compositions as described herein. A variety of conditions may be treated with the present methods and they include, but are not limited to ocular conditions, osteoarthritis, radiculopathy, spondylitis, and spondylosis. The compositions may, according to in one embodiment, be injected into a patient at a location such as a peripheral location.

Rate of release of at least one target solute such as triamcinolone acentonide may be controlled, according to one embodiment, by adjusting the porosity of the possaccharide's matrix. The adjusting step includes, but are not limited to, altering the polysaccharide's concentration, degree of cross-linking, molecular weight distribution or cross-linking agents. The parameters may be adjusted alone or in combination. Further, reactions conditions affecting the porosity of polysaccharide matrix during cross-linking may be modified to achieve varying or desired rate of release.

The present disclosure also relates to pharmaceutical compositions which include the novel biocompatible polysaccharide gel formulation with a pharmaceutical carrier.

The advantages and features of the present compositions and methods as disclosed herein, will be made more apparent from the description and claims that follow.

### DETAILED DESCRIPTION OF THE INVENTION

One embodiment of the present disclosure relates to a method of producing a biocompatible polysaccharide gel composition having sustained release properties comprising grafting at least one target solute onto a polysaccharide by covalent linkage of the at least one target solute with the polysaccharide. Covalent bonding is a form of chemical bonding that is characterized by the sharing of pairs of electrons between atoms, or between atoms and other covalent bonds. In short, attraction-to-repulsion stability that forms between atoms when they share electrons is known as covalent bonding.

Covalent bonding includes many kinds of interactions, including σ-bonding, π-bonding, metal-metal bonding, agostic interactions, and three-center two-electron bonds. The term covalent bond dates from 1939. The prefix co—means jointly, associated in action, partnered to a lesser degree, etc.; thus a “co-valent bond”, essentially, means that the atoms share “valence”, such as is discussed in valence bond theory. In the molecule H₂, the hydrogen atoms share the two electrons via covalent bonding. Covalency is greatest between atoms of similar electronegativities. Thus, covalent bonding does not necessarily require the two atoms be of the same elements, only that they be of comparable electronegativity. Because covalent bonding entails sharing of electrons, it is necessarily delocalized. Furthermore, in contrast to electrostatic interactions (“ionic bonds”), the strength of covalent bond depends on the angular relationship between atoms in polyatomic molecules.

Grafting is achieved in the present disclosure by covalent linkage. Target solutes can be grafted into the polysaccharide network as a result of reactions for such linkage. They may be those based on acid base chemistry, with functional groups such as hydroxyl and carboxyl groups. The susceptible bonds include the hydroxyl and/or carboxyl groups of the polysaccharide (e.g., hyaluronic acid disaccharide). Breaking of these bonds in one embodiment permits the advantageous controlled and sustained release of at least one target solute.

A polysaccharide such as hyaluronic acid is a polymer and has hydroxyl and carboxyl functional groups which may be useful for such linkage. Covalent linkage of at least one target solute or drug can be done for example by acid/base reactions with such groups and the susceptible functional groups on at least one target solute such as triamcinolone acetonide.

One example of reactions which may be utilized to achieve covalent linkage is condensation. A condensation reaction is a chemical reaction in which two molecules or moieties (functional groups) combine to form one single molecule, together with the loss of a small molecule. When this small molecule is water, it is known as a dehydration reaction; other possible small molecules lost are hydrogen chloride, methanol, or acetic acid. When two separate molecules react, the condensation is termed intermolecular. A simple example is the condensation of two amino acids to form the peptide bond characteristic of proteins. This reaction example is the opposite of hydrolysis, which splits a chemical entity into two parts through the action of the polar water molecule, which itself splits into hydroxide and hydrogen ions. If the union is between atoms or groups of the same molecule, the reaction is termed intramolecular condensation, and in many cases leads to ring formation. An example is the Dieckmann condensation, in which the two ester groups of a single diester molecule react with each other to lose a small alcohol molecule and form a β-ketoester product.

In polymer chemistry, a series of condensation reactions take place whereby monomers or monomer chains add to each other to form longer chains. This may also be termed as ‘condensation polymerization’ or ‘step-growth polymerization’. It occurs either as a homopolymerization of an A-B monomer or a polymerization of two co-monomers A-A and B-B. Small molecule condensates are usually liberated, unlike in polyaddition where there is no liberation of small molecules. A high conversion rate is required to achieve high molecular weights as per Carothers' equation. In general, condensation polymers form more slowly than addition polymers, often requiring heat. They are generally lower in molecular weight. Monomers are consumed early in the reaction; the terminal functional groups remain active throughout and short chains combine to form longer chains. Bifunctional monomers lead to linear chains (and therefore thermoplastic polymers), but when the monomer functionality exceeds two, the product is a thermoset polymer.

Using a reaction such as condensation is within the scope and teachings of the present disclosure covalent link at least one target solute such a triamcinolone acetonide to a polysaccharide such as hyaluronic acid. Triamcinolone acetonide is a synthetic glucocorticoid corticosteroid with anti-inflammatory action and has the chemical name 9-Fluoro-11,21-dihydroxy-16,17-[1-methylethylidenebis(oxy)]pregna-1,4-diene-3,20-dione. Typically delivered via intravitreal injection, the ophthalmic indications for triamcinolone acetonide include sympathetic ophthalmia, temporal arteritis, uveitis, and ocular inflammatory conditions unresponsive to topical corticosteroids. These are inflammatory conditions that can result in vision loss.

Other corticosteroids may also be utilized as at least one target solute. Examples of useful corticosteroids include, without limitation, cortisone, prednesolone, triamcinolone, triamcinolone acetonide, fluorometholone, dexamethosone, medrysone, loteprednol, derivatives thereof and mixtures thereof. As used herein, the term “derivative” referes to any substance which is sufficiently structurally similar to the material of which it is identified as a derivative so as to have substantially similar functionality or activity, for example, therapeutic effectiveness, as the material when the substance is used in place of the material.

At least one target solute may be covalently linked to a polysaccharide such as hyaluronic acid or hyaluronate as already stated. It is also within the scope and teachings of the present disclosure to use other polysaccharide which have the necessary functional groups to covalent link at least one target solute such as a drug with it. These include but are not limited to dextran sulfate, chondroitin sulfate, dermatan sulfate, chitosan, keratin sulfate, heparin, heparin sulfate and alginate.

The polysaccharides utilized, such as hyaluronate, may be cross-linked or not cross-linked. Cross-linking may be done to varying degrees, thereby permitting alteration of characteristics such as rate of HA migration for the peripheral location of administration, rate of active agent diffusion and migration out of the HA carrier. With more cross-linking the hyaluronic acid will reside in a target area for a longer period of time. Additionally, although preferably the polymeric hyaluronate in triamcinolone acetonide (Trivaris®) is a non-cross linked hyaluronate (so as to obtain, upon application of force to the plunger of the syringe used to administer Trivaris®, a high shear rate and hence relative ease of injection of Trivaris® through a 27-33 gauge needle), the hyaluronate can alternately be a cross-linked hyaluronate (to form a true hydrogel therefore) with a significantly lower viscosity (i.e. with a viscosity of about 5,000 cps at a shear rate of about 0.1/second at about 25 degrees Celsius). Such a cross-linked hyaluronate can have the same or similar excellent corticosteroid suspension property of Trivaris®, and have the additional advantage of longer residency (i.e. biodegradable at a slower rate) of the hyaluronate in the peripheral, with resulting prolonged nominal immunogenicity of such a cross-linked hyaluronate formulation in the peripheral, due to a longer period of peripheral (or peripheral) retention of the corticosteroid particles in the polymeric matrix of the cross-linked hyaluronate. Cross-linked and non-cross linked hyaluronans can also be blended in various proportions to optimize syringeability while slowing biodegradation and improving long-term retention within inflammed tissues, such as in the treatment of osteoarthritis. Furthermore, besides cross-linked hyaluronate other cross-linked polymers can be used, such as for example a polycarbophil.

At least one target solute may be sustained released by associating it with hyaluronic acid. HA may surround at least one target solute which embeds it in its matrix. As described herein, a further controlling parameter is introduced with the present novel covalent linkage of at least one target solute with a polysaccharide such as hyaluronic acid. The formed covalent bonds may be broken by a reaction such as hydrolysis. The breaking of the covalent bonds release the target solutes so that they may perform the pharmaceutical functions they were intended for in the body of a patient.

Hydrolysis is a chemical reaction or process in which a chemical compound is broken down by reaction with water. This is the type of reaction that is used to break down polymers. Water is added in this reaction. In organic chemistry, hydrolysis can be considered as the reverse or opposite of condensation, a reaction in which two molecular fragments are joined for each water molecule produced. As hydrolysis may be a reversible reaction, condensation and hydrolysis can take place at the same time, with the position of equilibrium determining the amount of each product.

In a hydrolysis reaction that involves breaking an ester link, one hydrolysis product contains a hydroxyl functional group, while the other contains a carboxylic acid functional group. The carbonyl is attacked by a hydroxide anion (or a water molecule, which is rapidly deprotonated). The resulting tetrahedral intermediate breaks down. The alkoxide fragment breaks off from the tetrahedral carbon and becomes an alcohol by protonation, leaving the acyl fragment with the attacking hydroxide, to produce a carboxylic acid. This is the reverse of the esterification reaction, yielding the original alcohol and carboxylic acid again. In a basic solution, the carboxylic acid is deprotonated, such that the basic hydrolysis is irreversible, while acidic hydrolysis is not.

There are two main methods for hydrolyzing esters, basic hydrolysis and acid-catalysed. With acid-catalysed hydrolysis a dilute acid is used to protonate the carbonyl group in order to activate it towards nucleophilic attack by a water molecule. However the more usual method for ester hydrolysis involves refluxing the ester with an aqueous base such as NaOH or KOH. Once the reaction is complete, the carboxylate salt is acidified to release the free carboxylic acid.

Moreover, the polysaccharide into which at least one target solute can be grafted is cross-linked or uncrosslinked. Crosslinking of a polysaccharide can be done for example by acid base chemistries. The cross-linking reagents useful for crosslinking a polysaccharide such as hyaluronic acid include 1,4 Butanediol Diglycidal Ether or Divinyl Sulfone. For the presently disclosed methods of producing a biocompatible polysaccharide gel, the polysaccharide can include for example, but not limited to hyaluronic acid, dextran sulfate, chondroitin sulfate, dermatan sulfate, chitosan, keratin sulfate, heparin, heparin sulfate, and alginate.

The at least one target solute which is grafted onto the polysaccharide can be for example, a drug. The drug can be, but not limited to, triamcinolone acetonide. A drug, broadly speaking, is any chemical substance that, when absorbed into the body of a living organism, alters normal bodily function. It is a chemical substance used in the treatment, cure, prevention, or diagnosis of a disease or used to otherwise enhance physical or mental well-being.

Sustained-release as used herein includes extended-release (ER, XR, or XL), time-release or timed-release, controlled-release (CR), or continuous-release (CR) formulations dissolve slowly. Sustained release formulations release at least one target solute or drug over time. The advantages of sustained-release formulations are that they can often be taken less frequently than instant-release formulations of the same drug, and that they keep steadier levels of the drug in the bloodstream. Sustained-release formulations are made so that the active ingredient is embedded in a matrix of insoluble substance (various: some acrylics, even chitin) so that the dissolving drug has to find its way out through the holes in the matrix. In some sustained release formulations the matrix physically swells up to form a gel, so that the drug has first to dissolve in matrix, then exit through the outer surface.

Difference between controlled release and sustained release is that controlled release is perfectly zero order release, that is, the drug releases with time irrespective of concentration. On the other hand, sustained release implies slow release of the drug over a time period. It may or may not be controlled release.

Another aspect of the present disclosure relates to a method of producing a biocompatible polysaccharide gel composition comprising encapsulating at least one target solute into the porous network of a polysaccharide gel. A porous network can be associated with a polysaccharide. A polysaccharide which is a polymer made up of many monosaccharides joined together by glycosidic bonds can have spaces which are available for encapsulation of target solutes. The porous network of a polysaccharide allows for a sustained release of at least one target solute which has been encapsulated in the polysaccharide. For example, at least one target solute such as triamcinolone acetonide can be encapsulated in hyaluronic acid particles. Sustained release may be achieved by the at least one target solute making its way through the porous network.

For this method of producing a biocompatible polysaccharide gel composition comprising encapsulating at least one target solute into the porous network of a polysaccharide gel, the polysaccharide can be for example but not limited to: hyaluronic acid, dextran sulfate, chondroitin sulfate, dermatan sulfate, chitosan, keratin sulfate, heparin, heparin sulfate, and alginate. Also herein, the polysaccharide into which at least one target solute can be encapsulated can be cross-linked or not cross-linked. There are cross-linking reagents useful for crosslinking a polysaccharide such as hyaluronic acid. These include for example 1,4 Butanediol Diglycidal Ether or Divinyl Sulfone.

Further, a drug which is suitable for encapsulation into the polysaccharide can be, but not limited to, triamcinolone acetonide. Another aspect of the present disclosure relates to a biocompatible polysaccharide gel composition having sustained release properties comprising at least one target solute grafted onto a polysaccharide by covalent linkage of the at least one target solute with the polysaccharide. As is true for the associated methods for making the biocompatible polysaccharides gel compositions of the present disclosure, the polysaccharide utilized may be cross-linked or not cross-linked. Further, the polysaccharide utilized may be selected from the group consisting of hyaluronic acid, dextran sulfate, chondroitin sulfate, dermatan sulfate, chitosan, keratin sulfate, heparin, heparin sulfate, and alginate. A preferred embodiment is hyaluronic acid. The at least one target solute may be a drug such as triamcinolone acetonide.

Alternatively, a preferred biocompatible composition in accordance with the scope and teachings of the present disclosure is a biocompatible hyaluronic acid gel composition having sustained release properties which comprises triamcinolone acetonide grafted onto hyaluronic acid by covalent linkage of triamcinolone acetonide with the hyaluronic acid. For the biocompatible polysaccharide gel composition produced by the process comprising encapsulating at least one target solute into the porous network of a polysaccharide gel, the polysaccharide can be, for example: hyaluronic acid, dextran sulfate, chondroitin sulfate, dermatan sulfate, chitosan, keratin sulfate, heparin, heparin sulfate, and alginate. The at least one target solute which is grafted onto the polysaccharide can be for example, a drug. A drug as used herein refers to a chemical substance used in the treatment, cure, prevention, or diagnosis of disease or used to otherwise enhance physical or mental well-being. The drug can be, but not limited to, triamcinolone acetonide.

Another aspect of the present disclosure relates to a method of treating a disease or condition comprising administering a therapeutically effective amount of the composition of the present biocompatible polysaccharide gel formulations. An example of a diseases or condition is an ocular condition such as an inflammatory ocular condition which may be treated with Trivaris®. Examples of other ocular conditions within the scope and teachings of the present disclosure include sympathetic ophthalmia, temporal arteritis, and uveitis.

Retinal diseases that can potentially be treated with the scope and teachings of the present disclosure include wet and dry age related macular degeneration(AMD), diabetic macular edema, and retinal vein occlusion associated macular edema. Active pharmaceutical ingredients especially for choroidal neovascularization (CNV) include but are not limited to anti-VEGF compounds such as Avastin®, Lucentis® or other full-length monoclonal antibodies or antibody fragments. Others include anti-VEGF aptamers (e.g. Pegaptanib®), soluble recombinant decoy receptors (e.g. VEGF Trap), corticosteroids, small interfering RNA's decreasing expression of VEGFR or VEGF ligand, post-VEGFR blockade with tyrosine kinase inhibitors, MMP inhibitors, IGFBP3, SDF-1 blockers, PEDF, gamma-secretase, Delta-like ligand 4, integrin antagonists, HIF-1 alpha blockade, protein kinase CK2 blockade, and inhibition of stem cell (i.e. endothelial progenitor cell) homing to the site of neovascularization using vascular endothelial cadherin (CD-144) and stromal derived factor (SDF)-1 antibodies. Agents that have activity against CNV that are not necessarily anti-VEGF compounds can also be used and include anti-inflammatory drugs, rapamycin, cyclosporine, anti-TNF agents, and anti-complement agents. Anti-complement agents may also be very useful for treating all forms of dry AMD including geographic atrophy. Agents that are neuroprotective and can potentially reduce the progression of dry macular degeneration can be used, such as the class of drugs called the ‘neurosteroids.’ These include drugs such as dehydroepiandrosterone (DHEA) (Brand names: Prastera® and Fidelin®), dehydroepiandrosterone sulfate, and pregnenolone sulfate. Other neuroprotective agents can be used such as brimonidine and other alpha agonists, and CNTF. All of these ingredients or drugs or compounds may be utilized as one or more target solutes within the scope and teachings of the present disclosure.

Also disclosed herein are methods of controlling rate of release of at least one target solute from the presently disclosed biocompatible polysaccharide gel composition comprising the step of adjusting the porosity of the polysaccharide's matrix. The rate of release can be tuned by adjusting the porosity of the gel matrix by modulating the hindrance effect through alter certain parameters. These parameters include, polysaccharide (e.g. hyaluronic acid) concentration, degree of crosslinking, crosslinker chemistry, molecular weight distribution of raw material polysaccharide (e.g. hyaluronic acid) and reaction conditions that have a direct effect on overall porosity of the polysaccharide gel matrix during cross-linking. For example, employing or containing a sufficient concentration of high molecular weight sodium hyaluronate in the present gel compositions allow formation of viscous gelatinous plugs for administration.

Another aspect of the present disclosure relates to a pharmaceutical composition comprising the present biocompatible polysaccharide gel formulation and a pharmaceutical carrier. The pharmaceutical composition can optionally include one or more agents such as, without limitation, emulsifying agents, wetting agents, sweetening or flavoring agents, tonicity adjusters, preservatives, buffers or antioxidants. Tonicity adjustors useful in a pharmaceutical composition of the invention include, but are not limited to, salts such as sodium acetate, sodium chloride, potassium chloride, mannitol or glycerin and other pharmaceutically acceptable tonicity adjusters. Preservatives useful in the pharmaceutical compositions of the invention include, without limitation, benzalkonium chloride, chlorobutanol, thimerosal, phenyl mercuric acetate, and phenyl mercuric nitrate. Various buffers and means for adjusting pH can be used to prepare a pharmaceutical composition, including but not limited to, acetate buffers, citrate buffers, phosphate buffers and borate buffers. Similarly, antioxidants useful in pharmaceutical compositions are well known in the art and includes for example, sodium metabisulfite, sodium thiosulfate, acetylcysteine, butylated hydroxyanisole and butylated hydroxytoluene. It is understood that these and other substances known in the art of pharmacology can be included in a pharmaceutical composition of the invention. See for example, Remington's Pharmaceutical Sciences Mac Publishing Company, Easton, Pa. 16^{th} Edition 1980.

As used herein, “carrier,” “inert carrier,” and “acceptable carrier” may be used interchangeably and refer to a carrier which may be combined with the presently disclosed polysaccharide gel in order to provide a desired composition. Those of ordinary skill in the art will recognize a number of carriers that are well known for making specific remedial pharmaceutical compositions.

The present compositions may include one or more other components in amounts effective to provide one or more useful properties and/or benefits to the present compositions. For example, although the present compositions may be substantially free of added preservative components, in other embodiments, the present compositions include effective amounts of preservative components, preferably such components which are more compatible with or friendly to tissues into which the composition is placed than benzyl alcohol. Examples of such preservative components include, without limitation, benzalkonium chloride, chlorhexidine, PHMB (polyhexamethylene biguanide), methyl and ethyl parabens, hexetidine, chlorite components, such as stabilized chlorine dioxide, metal chlorites and the like, other ophthalmically acceptable preservatives and the like and mixtures thereof. The concentration of the preservative component, if any, in the present compositions is a concentration effective to preserve the composition, and is often in a range of about 0.00001% to about 0.05% or about 0.1% (w/v) of the composition.

In addition, the present composition may include an effective amount of resuspension component effective to facilitate the suspension or resuspension of the corticosteroid component particles in the present compositions. As noted above, in certain embodiments, the present compositions are free of added resuspension components. In other embodiments of the present compositions effective amounts of resuspension components are employed, for example, to provide an added degree of insurance that the corticosteroid component particles remain in suspension, as desired and/or can be relatively easily resuspended in the present compositions, such resuspension be desired. Advantageously, the resuspension component employed in accordance with the present invention, if any, is chosen to be more compatible with or friendly to the tissues into which the composition is placed than polysorbate 80.

Any suitable resuspension component may be employed in accordance with the present invention. Examples of such resuspension components include, without limitation, surfactants such as poloxanes, for example, sold under the trademark Pluronic®; tyloxapol; sarcosinates; polyethoxylated castor oils, other surfactants and the like and mixtures thereof.

One very useful class of resuspension components are those selected from vitamin derivatives. Although such materials have been previously suggested for use as surfactants in compositions, they have been found to be effective in the present compositions as resuspension components. Examples of useful vitamin derivatives include, without limitation, Vitamin E tocopheryl polyethylene glycol succinates, such as Vitamin E tocopheryl polyethylene glycol 1000 succinate (Vitamin E TPGS). Other useful vitamin derivatives include, again without limitation, Vitamin E tocopheryl polyethylene glycol succinamides, such as Vitamin E tocopheryl polyethylene glycol 1000 succinamide (Vitamin E TPGSA) wherein the ester bond between polyethylene glycol and succinic acid is replaced by an amide group.

The presently useful resuspension components are present, if at all, in the compositions in accordance with the present invention in an amount effective to facilitate suspending the particles in the present compositions, for example, during manufacture of the compositions or thereafter. The specific amount of resuspension component employed may vary over a wide range depending, for example, on the specific resuspension component being employed, the specific composition in which the resuspension component is being employed and the like factors. Suitable concentrations of the resuspension component, if any, in the present compositions are often in a range of about 0.01% to about 5%, for example, about 0.02% or about 0.05% to about 1.0% (w/v) of the composition.

Unless otherwise indicated, all numbers expressing quantities of ingredients, properties such as molecular weight, reaction conditions, and so forth used in the specification and claims are to be understood as being modified in all instances by the term “about.” Accordingly, unless indicated to the contrary, the numerical parameters set forth in the specification and attached claims are approximations that may vary depending upon the desired properties sought to be obtained by the present invention. At the very least, and not as an attempt to limit the application of the doctrine of equivalents to the scope of the claims, each numerical parameter should at least be construed in light of the number of reported significant digits and by applying ordinary rounding techniques. Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements.

The terms “a,” “an,” “the” and similar referents used in the context of describing the invention (especially in the context of the following claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein is intended merely to better illuminate the invention and does not pose a limitation on the scope of the invention otherwise claimed. No language in the specification should be construed as indicating any non-claimed element essential to the practice of the invention.

Groupings of alternative elements or embodiments of the invention disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Certain embodiments of this invention are described herein, including the best mode known to the inventors for carrying out the invention. Of course, variations on these described embodiments will become apparent to those of ordinary skill in the art upon reading the foregoing description. The inventor expects skilled artisans to employ such variations as appropriate, and the inventors intend for the invention to be practiced otherwise than specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

Furthermore, numerous references have been made to patents and printed publications throughout this specification. Each of the above-cited references and printed publications are individually incorporated herein by reference in their entirety.

In closing, it is to be understood that the embodiments of the invention disclosed herein are illustrative of the principles of the present invention. Other modifications that may be employed are within the scope of the invention. Thus, by way of example, but not of limitation, alternative configurations of the present invention may be utilized in accordance with the teachings herein. Accordingly, the present invention is not limited to that precisely as shown and described.

We claim:

1. A method of producing a biocompatible polysaccharide gel composition having sustained release properties comprising grafting at least one target solute onto a polysaccharide by covalent linkage of said at least one target solute with said polysaccharide.

2. The method of claim 1 wherein said covalent linkage is made with one or more hydroxyl and/or carboxyl groups of said polysaccharide.

3. The method of claim 1, wherein said polysaccharide is cross-linked.

4. The method of claim 1, wherein said polysaccharide is selected from the group consisting of hyaluronic acid, dextran sulfate, chondroitin sulfate, dermatan sulfate, chitosan, keratin sulfate, heparin, heparin sulfate, and alginate.

5. The method of claim 1, wherein said polysaccharide is hyaluronic acid.

6. The method of claim 1, wherein said at least one target solute is a drug.

7. The method of claim 6, wherein said drug is triamcinolone acetonide.

8. A method of producing a biocompatible polysaccharide gel composition comprising encapsulating at least one target solute into the porous network of a polysaccharide gel.

9. The method of claim 8, wherein said polysaccharide is cross-linked.

10. The method of claim 8, wherein said polysaccharide is selected from the group consisting of hyaluronic acid, dextran sulfate, chondroitin sulfate, dermatan sulfate, chitosan, keratin sulfate, heparin, heparin sulfate, and alginate.

11. The method of claim 8, wherein said polysaccharide is hyaluronic acid.

12. The method of claim 8, wherein said at least one target solute is a drug.

13. The method of claim 12, wherein said drug is triamcinolone acetonide.

14. A biocompatible polysaccharide gel composition having sustained release properties comprising at least one target solute grafted onto a polysaccharide by covalent linkage of said at least one target solute with said polysaccharide.

15. The biocompatible polysaccharide gel composition of claim 14, wherein said polysaccharide is cross-linked.

16. The biocompatible polysaccharide gel composition of claim 14, wherein said polysaccharide is selected from the group consisting of hyaluronic acid, dextran sulfate, chondroitin sulfate, dermatan sulfate, chitosan, keratin sulfate, heparin, heparin sulfate, and alginate.

17. The biocompatible polysaccharide gel composition of claim 14, wherein said polysaccharide is hyaluronic acid.

18. The biocompatible polysaccharide gel composition of claim 14, wherein said at least one target solute is a drug.

19. The biocompatible polysaccharide gel composition of claim 18, wherein said drug is triamcinolone acetonide.

20. A biocompatible hyaluronic acid gel composition having sustained release properties comprising triamcinolone acetonide grafted onto hyaluronic acid by covalent linkage of triamcinolone acetonide with said hyaluronic acid.

21. A method of treating a disease or condition comprising administering a therapeutically effective amount of the composition of claim 14 to a mammal in need thereof.

22. The method of claim 21, wherein said disease or condition is an ocular condition.

23. A method of controlling rate of release of at least one target solute from the biocompatible polysaccharide gel composition of claim 14 comprising the step of adjusting the porosity of said polysaccharide's matrix.

24. The method of claim 23, wherein said adjusting step comprises altering said polysaccharide's concentration, degree of cross-linking, molecular weight distribution, and cross-linking agents.

25. The method of claim 23, wherein said adjusting step comprises altering the degree of cross-linking of said polysaccharide.

26. The method of claim 23, wherein said adjusting step comprises altering the molecular weight distribution of said polysaccharide.

27. The method of claim 23, wherein said adjusting step comprises altering the reaction conditions affecting the porosity of said matrix during cross-linking.

28. A pharmaceutical composition comprising the biocompatible polysaccharide gel formulation of claim 14 and a pharmaceutical carrier.
